(19) United States
(12) Patent Application Publication
(10) Pub. No.: US 2024/0036057 A1
(43) Pub. Date: Feb. 1, 2024

(54) DNA APTAMERS FOR THE DETECTION OF MONOMERIC CRP

(71) Applicant: Universiteit Hasselt, Hasselt (BE)

(72) Inventor: Luc Michiels, Mol (BE)

(21) Appl. No.: 18/265,367

(22) PCT Filed: Dec. 9, 2021
(86) PCT No.: PCT/EP2021/085069

§ 371 Date: Jun. 5, 2023

(30) Foreign Application Priority Data
Dec. 9, 2020 (EP) 20212664.5

(51) Int. Cl. G01N 33/68; G01N 33/53; C12N 15/115

(52) U.S. Cl. CPC G01N 33/6863; G01N 33/5308; C12N 15/115; G01N 2333/4737; C12N 2310/16

(57) ABSTRACT

The present invention is directed to selective detection of monomeric C-reactive protein (CRP) in a body fluid sample. In particular, the present invention provides the use of a DNA aptamer for the detection of monomeric CRP in an isolated sample of body fluid from a subject. The invention also provides DNA aptamers that specifically bind to monomeric CRP. Finally, also a detection method for monomeric CRP in a body fluid sample using a DNA aptamer and a kit for the detection of monomeric CRP in a body fluid sample are described.

### FIELD OF THE INVENTION

The present invention is directed to selective detection of monomeric C-reactive protein (CRP) in a body fluid sample. In particular, the present invention provides the use of a DNA aptamer for the detection of monomeric CRP in an isolated sample of body fluid from a subject. The invention also provides DNA aptamers that specifically bind to monomeric CRP. Finally, also a detection method for monomeric CRP in a body fluid sample using a DNA aptamer and a kit for the detection of monomeric CRP in a body fluid sample are described.

### BACKGROUND TO THE INVENTION

C-reactive protein (CRP) is one of the most common biomarkers currently used in clinical practice for the diagnosis and prognosis of inflammation. For example, an elevated level of CRP in serum is a strong indicator of inflammation and the level increases up to 10,000-fold in response to acute infection. Controversial observations were made regarding the role of CRP in inflammation and even more pronounced in chronic inflammatory conditions, such as cardiovascular diseases (Ji et al., 2007, FASEB J, 21, 284-294). This contradictory role can be explained by recent findings that two conformations of the CRP protein exist (Eisenhardt et al., 2009, Cell Cycle 8:23, 3885-3892). The native pentameric CRP (pCRP), unlike other pentraxins, can be dissociated irreversibly to form a different isoform, monomeric CRP (mCRP) upon binding with liposomes, cell membranes and activated platelets through lysophosphatidylcholine. This conformational change is accompanied with a change in the inflammatory profile of the protein in which mCRP is acting pro-inflammatory and pCRP rather anti-inflammatory (Eisenhardt et al., 2009, Circulation Researc; 105:128-137).

Monomeric CRP (mCRP) is a non-soluble, membrane-bound, tissue ECM-associated pro-inflammatory protein which may induce localized inflammation (Sproston, N. R et al., 2018, Frontiers in Immunology 9: 754; Molins et al., 2008, Arteriosclerosis, Thrombosis, and Vascular Biology. 2008; 28:2239-2246 3). Upon binding to the Fcγ RI (CD64) and Fcγ RIII (CD16) receptor on monocytes, mCRP mediates a clear pro-inflammatory response (Habersberger et al., 2012, Cardiovascular Research, 96: 64-72). These observations indicate that mCRP may be a potential strong biomarker for the prognosis and diagnosis of the severity of acute myocardial infarction (AMI) patients (Wang et al., 2015; Atherosclerosis 239: 343-349). Notably, the mCRP is also a more potent activator of endothelial cells and neutrophils than pCRP (Khreiss et al., 2004; Circulation 109: 2016-2022). Moreover, mCRP regulates the complement pathway activation and the LDL metabolism in a more efficient and flexible fashion than pCRP (Ji et al., 2006, Arterioscler Thromb Vasc Biol, 26: 935-941; Biro et al., 2007, Immunology, 121: 40-50). Based on these observations, it seems that mCRP exhibits potent pro-inflammatory actions, whereas pCRP has anti-inflammatory properties and may serve as a precursor of pCRP (Khreiss et al., 2005, Circ Res, 97: 690-697; Trial et al., 2016, Inflamm Cell Signal 3(2): e1409). Therefore, mCRP could be a more sensitive indicator for the detection of early inflammation. However, commercially available antibodies (e.g. CRP-8 antibody) cross-react with pCRP (Schwedler et al., 2003, Nephrol Dial Transplant, 18: 2300-2307). It is uncertain whether the relevant findings can be attributed to pCRP or mCRP. Therefore, the establishment of a sensitive and specific tool for the detection of mCRP is needed to discriminate between the pro-inflammatory effects of mCRP and the anti-inflammatory effects of the pCRP isoform. Immuno-based methods using antibodies, such as ELISA, have been developed for the detection and quantification of CRP in liquid samples, though said methods are limited to the detection and quantification of CRP in general and they do not allow selective quantification of mCRP. In addition, classical antibodies as core elements in ELISA methods, both as immobilized and signalling agents, are expensive to produce and show immunogenic limitations. Aptamer-based detection methods are recently being developed as an alternative for antibody-based detection methods. Compared to antibodies, aptamers are smaller in size, can be easily modified, are cheaper to produce and can be generated against a wide array of target molecules. In particular, aptamers are single stranded RNA/DNA strands that are selected against a target protein by a SELEX (systemic evolution of ligands by exponential enrichment) process, in vitro. Aptamers serve as an alternative tool to antibodies, showing an enhanced specificity and stability. The application of aptamers in ELISA gives rise to an ELISA-derived assay, also called enzyme-linked apta-sorbent assay (ELASA) or aptamer-linked immobilized sorbent assay (ALISA).

An aptamer-based immune-detection method has previously been described for the detection of CRP. U.S. Pat. No. 8,624,008 describes the detection and quantification of CRP using a specific aptamer sequence. Though, this DNA aptamer does not allow the specific detection of monomeric CRP and is thus not suitable for the specific detection of mCRP.

Wang et al., 2011 discloses particular RNA-based CRP aptamers which are said to specifically bind to monomeric but not pentameric forms of CRP (Analytical and bioanalytical chemistry, vol. 401 no. 4, pg. 1309-1318). However, these experiments were performed in non-natural conditions, whereas the same aptamers tested in natural conditions (in a background of pCRP) were shown to bind pCRP both in this application as well as by others (see details in example 3)

The present invention describes a DNA aptamer-based diagnostic tool that allows the specific detection of monomeric CRP (mCRP) but not the pentameric CRP (pCRP). The present assay therefore offers a novel, low cost diagnostic platform for a reliable and specific detection of monomeric CRP only in human serum of for example patients with systemic inflammation.

### SUMMARY OF THE INVENTION

The present invention is directed to specific DNA aptamers for the selective detection and quantification of monomeric C-reactive protein (mCRP) in isolated body fluid samples. Typical for the present invention is that only mCRP is detected, whereas pentameric CRP is not. Therefore, in a first embodiment of the invention, the use of a DNA aptamer for the detection and/or quantification of mCRP in an isolated sample of body fluid from a subject is disclosed. In a further aspect, said use is characterized in that the DNA aptamer specifically binds to mCRP, and not to pCRP. Typical for the present invention is thus that said use specifically allows the detection and/or quantification of mCRP, whereas pCRP is not detected or quantified with the DNA aptamer as disclosed in the present invention.

In a further embodiment, the use of a DNA aptamer for the detection and/or quantification of mCRP is characterized in that the DNA aptamer comprises the following nucleotide sequence: 5′-kg rss ksk krs srd drk dkk rkd rwr vdv kkg dkr gtk-3′ (SEQ ID NO: 1). In said nucleotide sequence, k is a nucleotide selected from t or g; r is a nucleotide selected from a or g; s is a nucleotide selected from c or g; d is a nucleotide selected from t or g or a; w is a nucleotide selected from t or a; and v is a nucleotide selected from a or c or g, according to the IUPAC codes. In still a further embodiment, the use of a DNA aptamer for the detection and/or quantification of mCRP is characterized in that the DNA aptamer comprises a nucleotide sequence selected from 5′-tg gcg ggt tgt gaa ggg tgg agt atg gtc gtg ttg gtt-3′ (SEQ ID NO: 2); or 5′-gg agc gcg ggg gag agt agt ggg gaa cgg tgg aga gtt-3′ (SEQ ID NO: 3); or 5′-gg agg tgt gaa cgt tat gtg gta gag aga tgg gtg gtg-3′ (SEQ ID NO: 4). Even more specifically, the use of a DNA aptamer for the detection and/or quantification of mCRP is characterized in that the DNA aptamer comprises the nucleotide sequence 5′-tg gcg ggt tgt gaa ggg tgg agt atg gtc gtg ttg gtt-3 (SEQ ID NO: 2).

In a further aspect, the present invention provides a DNA aptamer that specifically binds to mCRP and comprises the following nucleotide sequence: 5′-kg rss ksk krs srd drk dkk rkd rwr vdv kkg dkr gtk-3′ (SEQ ID NO: 1). This DNA aptamer is typically characterized in that it does not bind to pCRP.

In a further embodiment, the DNA aptamer according to the present invention comprises a nucleotide sequence selected from 5′-tg gcg ggt tgt gaa ggg tgg agt atg gtc gtg ttg gtt-3′ (SEQ ID NO: 2); or 5′-gg agc gcg ggg gag agt agt ggg gaa cgg tgg aga gtt-3′ (SEQ ID NO: 3); or 5′-gg agg tgt gaa cgt tat gtg gta gag aga tgg gtg gtg-3′ (SEQ ID NO: 4). In still a further embodiment, the DNA aptamer according to the present invention comprises a nucleotide sequence 5′-tg gcg ggt tgt gaa ggg tgg agt atg gtc gtg ttg gtt-3′ (SEQ ID NO: 2). In another embodiment, the DNA aptamer according to the present invention comprises a nucleotide sequence selected from 5′-gg agc gcg ggg gag agt agt ggg gaa cgg tgg aga gtt-3′ (SEQ ID NO: 3). In still another embodiment, the DNA aptamer according to the present invention comprises a nucleotide sequence selected from 5′-gg agg tgt gaa cgt tat gtg gta gag aga tgg gtg gtg-3′ (SEQ ID NO: 4). In a most preferred embodiment, the DNA aptamer according to the present invention comprises a nucleotide sequence 5′-tg gcg ggt tgt gaa ggg tgg agt atg gtc gtg ttg gtt-3′ (SEQ ID NO: 2).

In a further embodiment of the invention, the aptamer of the present invention or as used in the present invention has a 5′ end modified by a thiol group, an amine group, a biotin, a fluorescent label, a luminescent label, an enzyme or a nanoparticle, such as for example a gold nanoparticle.

In a further aspect, the present invention provides a method for the detection of mCRP in body fluid sample. Said method is typically characterized in that it allows for the selective detection of mCRP whereas pCRP is not detected. Said method comprises the following: providing a DNA aptamer according to any of the embodiments as described above, contacting an isolated body fluid sample with said DNA aptamer in a binding assay, determining whether or not the DNA aptamer has bound to mCRP in said body fluid sample, wherein binding of the DNA aptamer to mCRP confirms the presence of mCRP in the body fluid sample.

In a further embodiment, the present invention provides also a method for the quantification of mCRP in an isolated body fluid sample. Said method comprises the following: providing a DNA aptamer according to any of the embodiments as described herein, contacting the body fluid sample with said DNA aptamer in a binding assay, determining whether or not the DNA aptamer has bound to mCRP in the body fluid sample, with binding of the DNA aptamer to mCRP confirming the presence of mCRP in the body fluid sample and wherein binding of the DNA aptamer to mCRP also provides an indication about the amount of mCRP in the body fluid sample. In a further embodiment, said quantification is performed using a standard curve prepared with known amounts of mCRP.

In still a further embodiment, the binding assay of the detection and/or quantification method of the present invention is selected from ELISA, ELASA, ALISA, surface plasmon resonance assay, capillary electrophoresis-laser-induced fluorescence (CE-LIF) assay, immunohistochemistry, (nano) fluorescence-activated cell sorting (FACS), or fluorescence correlation spectroscopy.

In another aspect, a kit is provided for the detection and/or quantification of mCRP in a body fluid sample. Said kit comprises a DNA aptamer according to any of the embodiments as described herein above, a blocking buffer, an aptamer binding buffer and a conjugate buffer.

In a further aspect, the body fluid sample as described in all different embodiments of the invention is selected from blood, serum, plasma, urine or saliva.

In light of the foregoing, the DNA aptamer of the invention is capable of specifically binding to mCRP, and the detection method and quantification method for mCRP according to the present invention thus has a high sensitivity and a high specificity.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1 Binding sensogram of antibodies 3H12 and 9C9 to mCRP on a CM5 chip (A); binding sensogram of aptamers AmC2, AmC3, AmC4 to mCRP on a CM4 chip (B); Binding response of antibodies (3H12,9C9) and aptamer (AmC2, AmC3, AmC4) to mCRP and pCRP (C).

FIG. 2 Single cycle kinetics analysis of 200s association and 200s dissociation of different concentrations (1-2-4-5-10 ug/ml) of mCRP on 3H12 coated CM5 chip (A). Multi-cycle kinetics analysis of 200s association and 200s dissociation of different concentration of AmC2 aptamer (50-100-200-500 nM) in aptamer binding buffer on a mCRP coated CM4 chip (B). Data fitting with a mathematical 1:1 Langmuir model (chi-square value=2RU²) is indicated in (C).

FIG. 3 Electrophoretic mobility shift assay. Free aptamer AmC2-488 and internal standard (IS)-488 (A), aptamer AmC2-488 incubated with pCRP (B), aptamer incubated with mCRP (100 ng/ml) and free AmC2-488 (C), aptamer incubated with mCRP (200 ng/ml) (D), aptamer incubated with mCRP (500 ng/ml) (E). Quantification of binding of aptamer AmC2 with mCRP based on area under the curve (AUC). The AUC of free aptamer decrease on incubation with mCRP (F). The affinity of aptamer specific to mCRP not pCRP based on peak and RFU measurement.

FIG. 4 Aptamer based ELISA analysis of free mCRP on surface with different AmC2 concentrations in aptamer binding buffer. The data was measured in three independent experiments under identical condition.

FIG. 5 The mCRP concentration measured in HUVEC supernatant of untreated and TNF-alpha treated conditions. The samples were tested in Duplo measurment (n=36). The inflamed treated samples measured significantly increase mCRP concentration in comparison to untreated samples (p value=2.2e-16).

FIG. 6 The mCRP serum concentration measured in in healthy controls (HC; n=43), in obese healthy controls (BS; n=28) and in patients with rheumatoid arthritis (RA; n=30) show a highly significant increase in RA patients as compared to the healthy controls (RA: p=1.5e-05; BS: p=0.018).

FIG. 7 The mCRP and pCRP concentrations measured in serum of stable Chronic Obstructive Pulmonary Disease (COPD; n=38) patients as compared to matched non-COPD control persons (CP; n=18) showing a highly significant in mCRP serum levels in COPD patients as compared to control persons (p<0.001).

FIG. 8: Typical dilution series curve for aptamer-mCRP (1:1) interaction as obtained for both AmC2 and RNA aptamers.

### DETAILED DESCRIPTION OF THE INVENTION

The present invention specifically provides DNA aptamers and use of said DNA aptamers for the specific detection mCRP in a body fluid sample. Until now, presently available immune-based methods only allowed for the detection of CRP, which is the pentameric form of CRP, and said methods were not able to differentiate between pCRP and mCRP in a body fluid sample. The inventors of the present invention surprisingly identified a set of DNA aptamers that specifically bind to mCRP, whereas said DNA aptamers do not bind to pCRP. Therefore, in some embodiments of the invention, the use of a DNA aptamer for the detection and/or quantification of mCRP in an isolated sample of body fluid is provided.

Throughout this document, the term “aptamer(s)” or “aptamer sequence(s)” refer to single-stranded nucleic acid molecules that show high-affinity binding to a target molecule such as a protein, polypeptide, lipid, glycoprotein, glycolipid, glycopeptide, saccharide, or polysaccharide. This single-stranded nucleic acid is ssDNA, RNA or derivatives thereof. The aptamer as used in the present invention is a ssDNA or a derivative thereof. The aptamer comprises a three-dimensional structure held in certain conformation(s) that provide intermolecular contacts to specifically bind its given target. Although aptamers are nucleic acid based molecules, the binding to the target molecule is not entirely dependent on a linear base sequence, but rather a particular secondary/tertiary/quaternary structure. The term also covers next generation aptamers such as X aptamers that can't be typically amplified by PCR but can be adapted by adding a link primer. Such aptamers can specifically bind to proteins of interest but can also be easily amplified, sequenced etc. in downstream process. The term also covers aptamers that include modified bases. It is envisaged that the aptamers may include traditional aptamers of 15 to 120 bases in length, as well as longer aptamers of approx. 200 bases in length.

Aptamers are thus short, 15-200 nucleotide single-stranded DNA or RNA sequences or proteins that bind to target molecules with high affinity and specificity through their 3-dimensional structures. Nucleic acid aptamers are often identified using an iterative enrichment technique, such as Systematic Evolution of Ligands by Exponential Enrichment (SELEX), where oligos or proteins with increased affinity and specificity to a target molecule are isolated from a sequence pool after several rounds of selection.

Because aptamers have similar target binding affinities for their targets as antibodies, yet offer several advantages over antibody-based affinity molecules, aptamers are often used as substitutes for antibodies. Aptamers are typically easier to produce, especially on large scale. They are physically more stable, and modifications that increase their intracellular stability are easily incorporated, all at a lower cost. Aptamers are easily purified and typically have low immunogenicity. They penetrate tissues to reach their target sites faster and more effectively than antibodies, due to their smaller size, and they are also able to target molecules with low antigenicity. Advantages of using aptamers therefore include that they don't require fabrication in cells or animals, which results in them being cost effective to manufacture. They exhibit minimal differences between batches and they are not easily influenced by environmental factors, such as external temperature, humidity, and as such aptamers can be stored easily.

The present invention thus provides DNA aptamers and their use for the specific detection and/or quantification of mCRP. Said DNA aptamers are characterized in that they comprise the following nucleotide sequence: 5′-kg rss ksk krs srd drk dkk rkd rwr vdv kkg dkr gtk-3′ (SEQ ID NO: 1). In said nucleotide sequence, k is a nucleotide selected from t or g; r is a nucleotide selected from a or g; s is a nucleotide selected from c or g; d is a nucleotide selected from t or g or a; w is a nucleotide selected from t or a; and v is a nucleotide selected from a or c or g, according to the IUPAC codes. Nucleotides in the sequences as used in the present application are referred to by their commonly accepted single-letter codes. In the sequences with optional nucleic acids at some positions, the one-letter symbols recommend by the IUPAC-IUB Biochemcial Nomenclature Commission are used. In particular, k represents a nucleotide selected from t or g; r represents a nucleotide selected from a or g; s represents a nucleotide selected from c or g; d represents a nucleotide selected from t, g or a; w represents a nucleotide selected from t or a; v is represents a nucleotide selected from a, c or g.

In a more preferred embodiment, the DNA aptamer comprises a nucleotide sequence selected from 5′-tg gcg ggt tgt gaa ggg tgg agt atg gtc gtg ttg gtt-3′ (SEQ ID NO: 2); or 5′-gg agc gcg ggg gag agt agt ggg gaa cgg tgg aga gtt-3′ (SEQ ID NO: 3); or 5′-gg agg tgt gaa cgt tat gtg gta gag aga tgg gtg gtg-3 (SEQ ID NO: 4). In a most preferred embodiment, the DNA aptamer comprises a nucleotide sequence which is 5′-tg gcg ggt tgt gaa ggg tgg agt atg gtc gtg ttg gtt-3′ (SEQ ID NO: 2).

In an even more preferred embodiment, the DNA aptamer is a DNA aptamer with a nucleotide sequence selected from 5′-gca cca gca tat tcg att gtg gcg ggt tgt gaa ggg tgg agt atg gtc gtg ttg gtt ggg cta gta ggt gca tca g-3′ (SEQ ID NO: 5); or 5′-gca cca gca tat tcg att ggg agc gcg ggg gag agt agt ggg gaa cgg tgg aga gtt ggg cta gta ggt gca tca g-3′ (SEQ ID NO: 6); or 5′-gca cca gca tat tcg att ggg agg tgt gaa cgt tat gtg gta gag aga tgg gtg gtg ggg cta gta ggt gca tca g-3′ (SEQ ID NO: 7). In a most preferred embodiment, the DNA aptamer is a DNA aptamer with a nucleotide sequence that is 5′-gca cca gca tat tcg att gtg gcg ggt tgt gaa ggg tgg agt atg gtc gtg ttg gtt ggg cta gta ggt gca tca g-3′ (SEQ ID NO: 5).

The DNA aptamer according to the different embodiments of the invention are thus capable of specifically binding mCRP without binding to pCRP. In other words, the DNA aptamers of the present invention and mCRP have high affinity and high specificity between each other. Therefore, the DNA aptamers of the present invention are particularly suitable for the specific detection of mCRP.

In some aspects of the invention, the DNA aptamers can be modified on their 5′ end by a thiol group, an amine group, a biotin, a fluorescent label, a luminescent label, an enzyme or a nanoparticle. This modification allows the DNA aptamer to be detected after binding to mCRP. The fluorescent label includes chemical substances such as fluorescein isothiocyanate (FITC), Cy3, and Cy5. The luminescent label includes chemical compounds such as acridinium esters, and enzymes may include alkaline phosphatase, horse radish peroxidase (HRP), and the like. The nanoparticle can be for example a gold nanoparticle.

It should be noted that other than using the aptamer of the invention for detecting and/or quantification of mCRP, the high affinity and high specificity between the aptamer and mCRP can also be applied in other biotechnologies. For example, the aptamer can be adopted as a target drug for carrying drugs or directly approaching a site with high expression of the mCRP to bind with mCRP, so as to release drugs or inhibit the expression of the mCRP directly, thereby treating or preventing diseases related to the expression of mCRP. Obviously, other than applying the aptamer for detection or as the target drug, skilled persons should understand that the aptamer of the invention is also suitable for other biotechnologies relying on the high affinity and high specificity of mCRP, and the details are thus not illustrated herein.

The present invention is further also directed to a method for the detection mCRP in an isolated body fluid sample. In said method, a DNA aptamer as described herein is provided and the body fluid sample and said DNA aptamer are mixed and contacted with each other in a binding assay. This allows the mCRP in the sample to bind to the DNA aptamer and in this way a mCRP-aptamer complex is formed. The mCRP or the aptamer in the mCRP-aptamer complex is then detected. In another embodiment, a method for the quantification of mCRP in an isolated body fluid sample is provided. In this method, a DNA aptamer as described herein is provided and the body fluid sample and said DNA aptamer are mixed and contacted with each other in a binding assay. This allows the mCRP in the sample to bind to the DNA aptamer and in this way a mCRP-aptamer complex is formed. The mCRP or the aptamer in the mCRP-aptamer complex is then detected and quantified. In a particular embodiment, the quantification of mCRP can be performed using different standards of mCRP and generating a standard curve. In another embodiment, quantification of mCRP is performed using different dilutions of the DNA aptamer to generate a standard curve. In a further aspect, the binding assay as used in any of the methods of the present invention, is selected from ELISA, ELASA, surface plasmon resonance assay, capillary electrophoresis-laser-induced fluorescence (CE-LIF) assay, immunohistochemistry, (nano) fluorescence-activated cell sorting (FACS), or fluorescence correlation spectroscopy.

In another embodiment, the detection method for mCRP is, for example, a sandwich ELISA method. In said method, a 5′ end of each of the DNA aptamers is modified by a biotin, for example. DNA aptamers and beads with modified streptavidins on their surface are non-covalently bonded, so that the DNA aptamers are connected to said streptavidin beads. In this step, since the biotins and the streptavidins have high affinity with each other, the DNA aptamers and the beads can rapidly bond to form a plurality of DNA aptamer-bead complexes, where the non-covalent bonds between the DNA aptamers and the bead are not easily affected by pH, temperature, organic solvent, or denaturant. In other embodiments, the DNA aptamers and the beads can also be bound by bonding methods other than the biotin streptavidin, and the invention is not limited thereto. The beads connected with the DNA aptamers and a series of diluted mCRP standard solutions or samples are mixed in an Eppendorf or in a multiwell plate to undergo binding. In this step, since the DNA aptamers have high affinity and specificity toward mCRPs, the DNA aptamers on the beads bind with the mCRPs in the mCRP standard solution or the sample to form mCRP-aptamer-bead complexes. Thereafter, mCRP bound to the DNA aptamers can be detected.

In another embodiment, a detection and/or quantification method for mCRP is for example a surface plasmon resonance (SPR) bio-sensing method. In said SPR method, DNA aptamers of the invention are coated on a metal thin film surface. Then, mCRPs in a sample bind to said DNA aptamers. In this step, the binding of mCRP and the DNA aptamers leads to a change in a resonance angle, such that the complete process of affinity reactions such as the binding and the dissociated between the DNA aptamers and the mCRPs in the sample can be obtained by detecting the change of the resonance angle. In the present embodiment, as the DNA aptamers and the mCRPs have high affinity and high specificity, the DNA aptamers are capable of capturing and then binding with the mCRPs in the sample, so as to detect the mCRPs through the change of the resonance angle. In addition, each being a DNA fragment, the DNA aptamers are not easily influenced by environmental factors such as external temperature, humidity, and the like. The detection and quantification methods for mCRP in these embodiments, therefore have high sensitivity, high stability, and high accuracy.

It should be noted that although in the embodiments aforementioned, the mCRPs of the invention are applied in ELISA methods or SPR methods as examples, the detection and quantification method for mCRPs in the present invention is not limited thereto. In other words, as the DNA aptamers of the invention have high affinity and high specificity for mCRP, the DNA aptamers can be applied in any detection method for detecting mCRP. In particular, since the DNA aptamers of the invention can replace the mCRP antibodies, the detection method for mCRP in the invention can be adopted in any detection method using binding principle of the mCRP antibodies and the antigens. These methods should be well known to those skilled in the art and are not described in detail hereinafter.

In the following, examples are provided to illustrate the methods for screening the DNA aptamers of the invention, verify the high affinity and specificity of the DNA aptamers toward the mCRPs, and to depict practical application of the detection method for mCRPs in the invention. The following illustrations are provided to describe the invention in detail for the implementation of persons skilled in the art, and not used to limit the scope of the invention.

### EXAMPLES

### Example 1: Development of mCRP Specific Aptamers

### Materials and Methods

mCRP Specific Aptamers and Antibodies

The mCRP specific antibody clones 3H12 and 9C9 against the target monomeric CRP were received from Prof. Lawrence Potempa (Roosevelt University, Chicago, US). A library with DNA aptamers that was developed using SELEX technology was purchased from Integrated DNA technologies and the following 3 aptamer sequences were used in this example: Aptamer AmC2: 5′-gca cca gca tat tcg att gtg gcg ggt tgt gaa ggg tgg agt atg gtc gtg ttg gtt ggg cta gta ggt gca tca g-3 (SEQ ID NO: 5) Aptamer AmC3: 5′-gca cca gca tat tcg att ggg agc gcg ggg gag agt agt ggg gaa cgg tgg aga gtt ggg cta gta ggt gca tca g-3′ (SEQ ID NO: 6) Aptamer AmC4: 5′-gca cca gca tat tcg att ggg agg tgt gaa cgt tat gtg gta gag aga tgg gtg gtg ggg cta gta ggt gca tca g-3 (SEQ ID NO: 7) All aptamers and labelled aptamers were purchased from Integrated DNA technologies (IDT).

The EDC (cat-77149) and NHS (cat-24500) were bought from Thermo Scientific, Belgium. SPR sensor chips were all purchased from Biacore. The human pCRP was purchased from BBi solutions, UK (Cat-P100-0). TMB substrates were purchased from Bio-Rad (cat-BUF054A).

Preparation of mCRP

Monomeric CRP was prepared by heat treatment of the commercially available human pCRP (BBi solutions, UK). Briefly a Slide-a-Lyzer G2 dialysis cassette Thermofisher Scientific) membrane was hydrated by immersing the cassette in a beaker containing dialysis buffer (137 mM NaCl, 2.7 mM KCl, 4.3 mM Na₂HPO₄, 1.47 mM KH₂PO₄ pH 7.2+0.1% NaN₃) for 2-3 min. Human pCRP purchased from Sigma is injected into the cassette at the inlet portal. The sample loaded cassette was dialysed in a dialysis buffer (300× of sample volume) 3 times for 2h at room temperature and additionally overnight at 4° C. After overnight dialysis, the sample was recovered through the unused needle port and incubated at 70° C. for one hour which results in monomerization of the pCRP and a change in its solubility. The monomerization process was finalized by adding 10 μl 1 M NaOH, and the recovered mCRP was stored at 4° C. for further experiments.

### Affinity Measurement Using Surface Plasmon Resonance (SPR)

The binding interaction between anti-mCRP antibodies, aptamer, target protein mCRP and pCRP was performed on a Biacore T200 SPR device (Biacore Uppsala, Sweden). All reagents used for the SPR analysis were purchased from Biacore.

mCRP antibodies (3H12/9C9) were immobilized on a CM5 sensor chip (GE Healthcare, Sweden) according to the standard Biacore protocol of the amine-coupling method. Briefly, the CM5 sensor chip was activated using freshly prepared 1M N-(3-dimethylaminopropyl)-N-ethylcarbodiimide hydrochloride (ECD) and 0.25 M N-hydroxysuccinimide (NHS) at 1:1 ratio in 1×HBS-N (0.1 M HEPES, 1.5 M NaCl, pH 7.4) as a running buffer for 420 sec. Then, 67 ng/ml of corresponding antibodies and a reference antibody (anti-QuinA, Apdia, Belgium) in sodium acetate buffer, pH=4, were immobilized on the activated carboxyl surface of CM5. The unbound residual carboxyl groups were then quenched with 1 M ethanolamine, pH=8 for 420 sec. After equilibrating the chip surface with the running buffer (PBS, pH 7.2), the binding affinities of the antibodies towards target proteins was determined by injecting 400 μl of 20 ng/ml mCRP for 700 sec at a flow rate of 30 μl/min.

For the SPR analysis of aptamers, mCRP and pCRP (45 nM) in sodium acetate buffer (pH 5) were first immobilized on the active flow cell and reference flow cell respectively on a CM4 sensor chip according the standard amine-coupling protocol of Biacore (EDC/NHS activation (1:1 ratio) in 1×HBS-N (0.1 M HEPES, 1.5 M NaCl, pH 7.4 as a running buffer for 420 sec). The unbound activated surface was further blocked by 1 M ethanolamine, pH 8 for 420 sec. The binding affinity between aptamers and mCRP was evaluated by injecting 200 nM of aptamers in 1× aptamer binding buffer (75 mM HEPES, 0.15 M NaCl, pH 7.4) for 420 sec with a flow rate of 30 μl/min. Regeneration of the CM4 chip flow cells was achieved by applying 10 mM NaOH with 120 sec followed by several washing steps with 1× aptamer binding buffer. SPR binding concentrations were obtained as a resonance unit RU (1 RU=1 pg/mm² of the sensor surface) by calculating the binding units of antibody/aptamer and mCRP and correcting for the background signal observed in the reference channel.

### Kinetic Study of Antibody 3H12 and Aptamer AC2

SPR was applied in a single cycle kinetics approach to analyze the kinetic constant of the 3H12 antibody for mCRP. Although they showed a high affinity for mCRP, due a stability issue of this antibody upon regeneration after immobilisation, this method was chosen. To achieve this, the reference flow cell is immobilized with a reference antibody anti-Quin and the active flow cell with 3H12. The kinetic analysis was performed with an increasing concentration of mCRP (0.6-10 pg/ml) in PBS with 200 sec of association and 200 sec of dissociation at flow rate of 30 μl/min and last step with regeneration 120 sec, 2 M NaOH.

On the contrary the aptamer was very stable regeneration steps and therefore a multicycle kinetics analysis was performed for the evaluation of reaction kinetics of the aptamer. Pentameric CRP is immobilized in the reference flow cell and mCRP in the active cell at a low immobilization level (70 RU) to achieve an optimal kinetic analysis on CM4 biosensor chips. The kinetic analysis was performed with increasing concentrations of aptamer AmC2 (0-500 nM) applying for 200 sec of association and 200 sec dissociation at flow rate of 300 μl/min. A regeneration step of 120 sec using 1 M NaOH was applied between the injections of different concentrations. The Biacore evaluation software was used in the analysis for estimation of affinity rate constant by fitting the obtained binding sensogram using standard mathematical 1:1 Langmuir model.

### Capillary Electrophoresis (CE)

All CE experiments were performed using a PA 800 plus electrophoresis system (Beckman coulter Inc.), combined with a Laser-induced fluorescence (LIF) detector with a 488 nm wavelength laser module (3 mW) excitation source and emission was monitored using a 520 nm band pass filter. Separation of all samples were performed using a base-fused silica capillary (Beckman Coulter, USA, cat-338451) with 50 um inner diameter and 375 um outer diameter and a total length of 67 cm. After rinsing the capillary with de-ionized water for 30 min, a capillary regeneration buffer (0.1N sodium hydroxide) was applied for 15 min. The sample injections column was rinsed with deionized water and running buffer for 3 min, separately. Labeled aptamer was pre-incubated with an increasing amount of mCRP for one hour in the presence of Alexa 488 labeled as internal standard (IS). After incubation, the samples were electrophoretically separated under the pressure 10 psi for 10 min. The electrophoresis data were collected and analyzed using the 32 Karat version 9.1 analysis package (Beckman coulter, Inc.,).

### ALISA Protocol

The selected AmC2 aptamer was used in the development of a diagnostic ALISA (Aptamer-Linked Immobilized Sorbent Assay). First, pCRP (2.5 pg/ml) was immobilized on COOH coated plates (Biomat, TN, Italy) by a standard covalent amine coupling using the EDC/NHS reaction. In brief, 0.4 M of EDC and 0.1 M of NHS was freshly prepared and mixed in a 1:1 ratio for activation of a COOH coated plate surface. After coating, the remaining uncovered surfaces and unbound residual activated carboxyl groups on the plates were blocked using 5% PBS-marvel and 1 M ethanolamine, pH 8 overnight and for 1 h respectively. Then, the in situ monomerization of pCRP to mCRP was achieved by applying 10 mM NaOH on the surface of coated plates for 30 min at RT. To obtain the optimal concentration for aptamer to interact with mCRP, 100 μl of freshly prepared biotin labeled aptamer in a serial dilution (0; 12.5; 25; 50; 100; 150; 200 nM) in aptamer binding buffer (ABB) was added to 25 μl of serum sample and incubated for 30 min. RT on a rotating mixer and then transferred to a mCRP coated plate for 30 min at 37° C. on a shaker at 400 rpm. After incubation, the wells were washed 3 times with ABB and incubated with SA-HRP (Life technologies, Belgium) in Aptamer Conjugation Buffer (ACB) for 30 min at 37° C. on a shaker at 400 rpm. After washing 3× with ABB for 5 min, TMB substrate was added for the colour development, subsequently the enzyme-substrate colour reaction was stopped by adding a stop solution (0.18 M H2SO4) to the wells. Optical density of standards and samples were measured using a Multiskan™ FC Microplate Absorbance Reader (Thermo Scientific, Belgium) at wavelength of 450 nm.

### In Vitro Samples Preparation

Human umbilical vein endothelial cells (HUVEC-2, BD Bioscience) at a density of 30000 cells were grown in EGM-2 MV medium supplemented with SingleQuot Kit (Lonza, USA) and 5% fetal bovine serum (Lonza, USA) in 24-well culture plates and used at passages 3-5. Cells reaching 70-75% confluency were washed with PBS (Lonza) and inflammation was then induced by adding 10 ng/ml TNF-α (Immunotools GmbH) in the refreshed medium overnight. The cell culture supernatants (SN) were collected from both untreated and inflamed cells and centrifuged at 2000 rpm for 10 min to remove the cell debris. The collected SN were stored at −20° C. for further experiments.

### Statistical Analysis

The data were represented as mean±standard deviation (SD), of three independent experiments. The significant difference between/within antibodies and aptamers were evaluated using one-way analysis of variance (ANOVA). The statistical significance between the healthy group vs. patients and in vitro samples healthy and inflamed supernatant were tested using two sample t-test using R version 4.0.1. P values<0.5 were considered statistically significant and p values>0.5 were considered statistically non-significant (ns).

### Results

Aptamers Specifically Bind with mCRP and not with pCRP

In order to assess the specificity and binding efficiency of both antibodies and aptamers for mCRP, we used a highly sensitive label-free SPR technique that enables to acquire semi-quantitative information on the specificity, sensitivity and affinity between an analyte and a ligand. The binding interaction is determined on the basis of the concentration of mass transition, which is directly proportional to the number of molecules bound to immobilized ligands. One response unit (RU) corresponds to 1 pg of protein bound per mm². The binding interactions were calculated in relation to the reference subtraction.

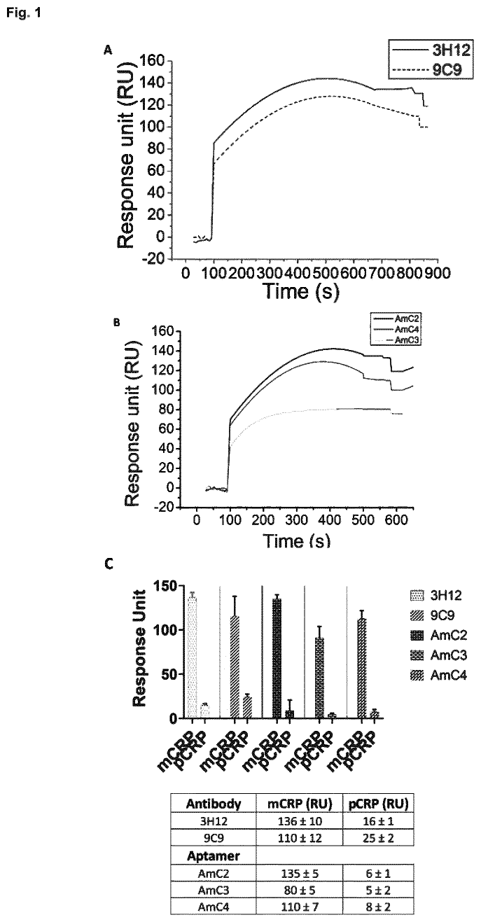

In this platform, the available antibodies against CRP (3H12, 9C9) and the anti-QuinA (as a negative control) were immobilized on CM5 chip using an amine coupling procedure with final immobilization level 1500 RU. The binding affinity of these antibodies was measured after running mCRP protein over the immobilized surfaces. The specific binding responses of mCRP towards clone 3H12 and 9C9 were 130±10 and 110±12RU, respectively while they show significantly lower interaction responses towards the pCRP 16±1 (p=6.76973E-14) and 25±2 RU (p=7.94E-08), respectively (FIG. 1 A, C). The binding response unit shows that the antibodies have significantly higher affinity to mCRP than to pCRP.

In the case of the aptamers, the monomeric and pentameric isoform of CRP (pCRP as a negative control) were immobilized on the surface of CM4 sensor chips at an immobilization level of ˜700 RU. The binding affinity of the aptamers was obtained by running different aptamers over mCRP and pCRP immobilized surfaces. The obtained binding resonance units for the aptamers AmC2 (A2), AmC3 (A3), AmC4 (A4) against mCRP were 135±5, 80±15 and 110±7 RU respectively, while against pCRP were 6±1 (p=6.01E-16), 5±2 (p=5.36E-10) and 8±2 (p=2.58E-12) respectively (FIG. 1 B, C). These results prove that the aptamers AmC2, AmC3, AmC4 are interacting significantly higher with mCRP and not pCRP. In addition, aptamer AmC2 showed a significantly comparable higher affinity for mCRP as compared to aptamers AmC3 (p value-6.41084E-07) and AmC4 (p value-1.990E-05) respectively. The clone 3H12 showed a higher affinity in comparison to 9C9 antibody but not highly significant (p value-0.01157 (NS)) Therefore, further kinetic investigations were focused on only the 3H12 antibody and AmC2 aptamer.

Binding Kinetics of Antibody and Aptamer Against Target mCRP

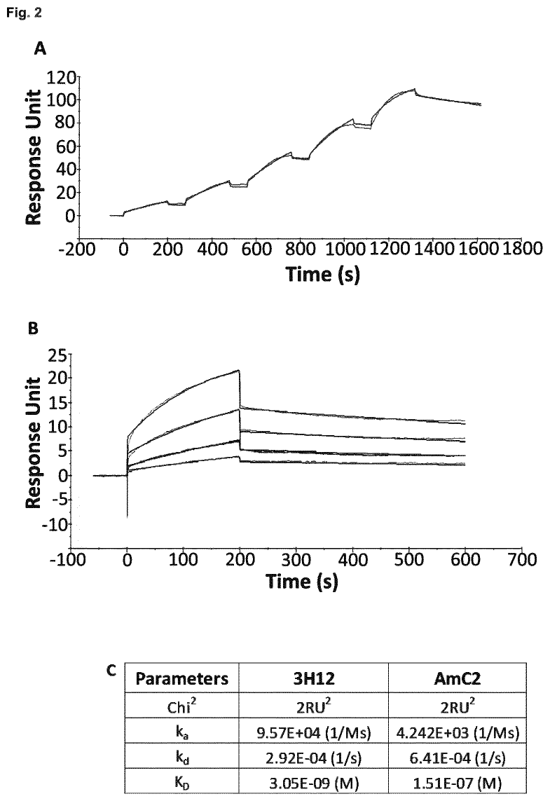

The kinetic parameters of the antibody 3H12 and aptamer AmC2 were evaluated using the inbuilt kinetics modules of the Biacore T200 SPR (FIG. 2). The kinetic analysis of biomolecular interactions can be studied by two different cycles, single cycle kinetics and multicycle kinetics. In multi cycle kinetics, different concentrations of the analyte are injected with a regeneration step between every sample injection, whereas in single cycle kinetics an increasing concentration of the analyte is injected sequentially ending with a final regeneration step. The single cycle kinetics approach is adopted when the ligand is difficult to regenerate or when regeneration is detrimental to the ligand. As the antibody clone 3H12 was not stable upon regeneration, a single cycle kinetics had to be applied. The kinetic analysis was performed using the Biacore T200 evaluation software working on mathematical 1:1 Langmuir model. The strength of the binding interaction between the ligand and target analyte can be measured based on the affinity constant value KD. The KD value refers to the proportion of a complex formed between ligand-analyte and the free interactants at equilibrium. The binding kinetics of the ligand to the target analyte is a time dependent interaction and in SPR this is measured as the association and dissociation constants (ka and kd). These constants reflect how the complex is formed or dissociating within a given time span. Fitting the antibody 3H12 binding curve with the model to obtain the best lowest chi-square value (2RU2), gives the following estimated constants ka=9.57E+04 (1/Ms), kd=2.92E-04 (1/s) and an affinity constant KD=3.05 nM (FIG. 2C). Fitting the aptamer AmC2 binding curve with the same 1:1 Langmuir mathematical model gave a best fit at a 0.171 RU2 chi-square value. The resulting estimated kinetic constants are ka=4.242E+03 (1/Ms), kd=6.41E-04 (1/s) and affinity an affinity constant KD=1.51E-07 (M) (FIG. 2C). Based on these results, the kinetic parameters of association and dissociation for both aptamer and antibody are in range to be implemented in an immunoassay technique, but the stability of the 3H12 antibody is problematic to be applied in an ELISA. Moreover, these antibody clones were developed against a mutated recombinant mCRP (rmCRP) whereas the aptamer was selected against native human mCRP.

Binding Affinity of Aptamer and mCRP Using Capillary Electrophoresis

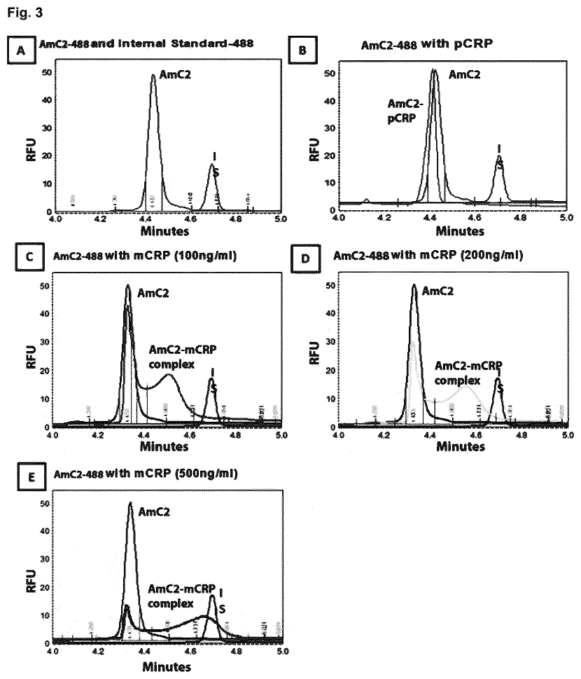

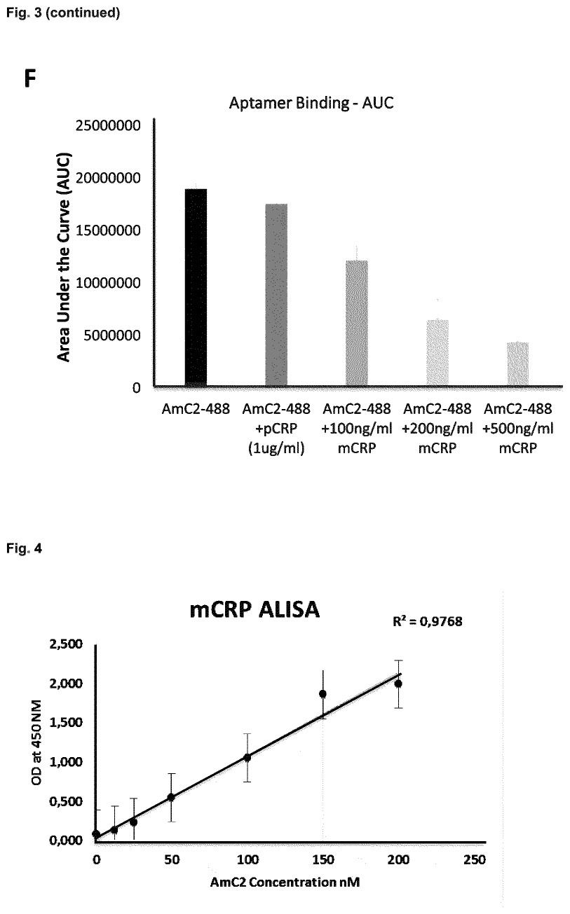

In laser induced fluorescence capillary electrophoresis systems (CE-LIF), the complex (aptamer-mCRP) and the free molecules (aptamer, mCRP) are separated based on their mass and charge. The aptamer-mCRP interaction is investigated using CE-LIF by combining the selective separation influence of electrophoretic mobility shift and is a rapid minimal sample consumption method. In this method, fluorescently labelled aptamer is titrated with an increasing concentration of the protein followed by an electrophoretic separation. The output is represented by two fluorescent peaks corresponding to the free aptamer and the aptamer-protein complex. The quantification of the assay is based on the area under the curve (AUC) of the peaks formed. FIG. 3 compares the electropherograms obtained for Alexa 488 labeled aptamer AmC2 (200 nM) at pH 7.4 in the presence and absence of mCRP or pCRP. As shown in the electropherograms (FIG. 3C, 3D, 3E) a mCRP concentration dependent decrease in the aptamer AmC2 peak-height is observed as compared to free aptamer AmC2 (FIG. 3A) clearly demonstrating the increased binding of mCRP to the aptamer AmC2. On the other hand no decrease in the aptamer peak-height was observed when incubated with pCRP (FIG. 3B) as compared to free aptamer AmC2 (FIG. 3A), showing that pCRP was not bound by the aptamer. Finally FIG. 3F shows the quantification of the assay with calculated AUC of free aptamer AmC2 that decreases significantly when incubated with increasing concentration of target mCRP and not pCRP confirming the aptamer-mCRP complex formation. The results prove the specificity of the aptamer AmC2 to mCRP and not pCRP which is in accordance with the SPR results.

mCRP Detection in the Supernatant of Endothelial Cells Undergoing Inflammation In Vitro

The characterized aptamer was implemented in the development of an immunoassay. In order to determine the optimal AmC2 concentration to be applied, the developed assay was tested for the detection of varying concentrations of biotin labelled aptamer in aptamer binding buffer. FIG. 4 shows the detection of varying concentrations of mCRP specific biotin aptamer. The increase in concentration showed a linear relationship with the OD measured at 450 nm (FIG. 4B). The optimal concentration for aptamer to interact with mCRP is detected at 150 nM beyond which saturation is observed (FIG. 4).

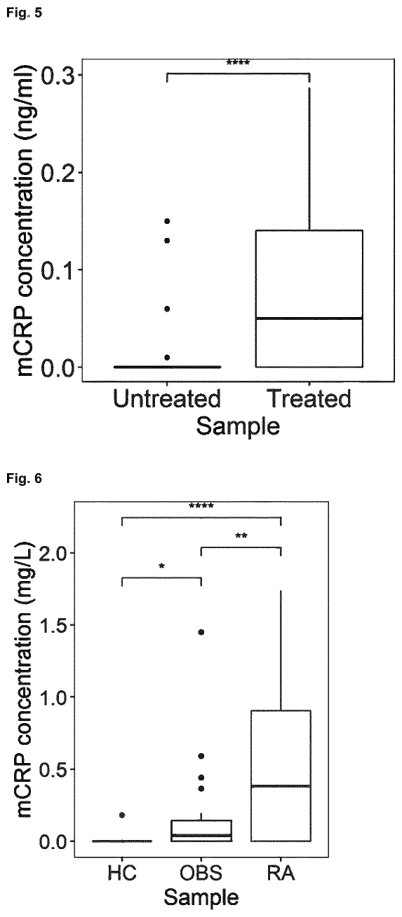

As a proof of principle the developed ALISA was applied to supernatant fluid of HUVEC cells that were either triggered or not for inflammation using TNF-α (FIG. 5). As expected a significant increase in the expression of mCRP could be detected in the supernatant of TNF-α treated HUVEC cells as compared to non-treated HUVEC (n=36; p=2.2e-16). The supernatant from cell free culture medium was used as negative control.

### Example 2: Detection of mCRP in Human Patient Samples

### Materials and Methods

### Patient Serum Samples

Rheumatoid Arthritis (RA) serum samples (n=30) were purchased from a commercial supplier ProMedx, USA. ProMedDx certified that the samples were collected in compliance with the 21 CFR 50/FDA guidance informed consent for use in medical research and deposited in the University Biobank Limburg (UBILIM, Jessa Hospital Belgium). Healthy controls (n=43) and samples from obese subjects (n=28) were received from UBILIM (Jessa Hospital). The study protocol was approved by the Medical Ethical Committee of Hasselt University (CME2018/033).

The patient samples and controls were replaced in-place of control serum in the developed mCRP ALISA and the chemiluminescence signal was measured at 450 nm.

In another experiment, pCRP and mCRP levels were assessed in serum samples of 38 patients with stable COPD.

This is an analysis of data collected at baseline of a prospective randomized controlled trial on the efficacy of a nutritional supplement in patients with COPD (NCT02770417). Patients were recruited at the outpatient consultation of Jessa Hospital (Hasselt, Belgium) and were clinically stable (e.g., X weeks exacerbation free). NCCP were recruited via advertisement within Hasselt University and Senior University of Hasselt University by staff members. Recruitment was performed between June 2016 and November 2018.

Age, gender, smoking status, number of hospitalizations<1 year, Charlson Comorbidity index, body mass index (BMI), fat mass index and lean mass index (fat mass/height²; lean mass/height²; DXA, lunar DPXL, General Electric Company GE, Boston, MA, USA), spirometry data (SpiroUSB, Carefusion, San Diego, CA, USA; according to American Thoracic Society/European Respiratory Society (ATS/ERS guidelines) from NCCP, pulmonary function testing data including post-bronchodilator spirometry, lung volumes and diffusion capacity for carbon monoxide (Master Screen Body and PFT, Jaeger, Carefusion, San Diego, CA, USA; according to ATS/ERS guidelines) from patients with COPD, impact of disease on daily life by COPD Assessment Test (CAT), grade of dyspnea by modified Medical Research Council (mMRC) scale, physical capacity (using the six-minute walk distance in meters), and physical activity (PA) as steps per day was assessed via a tri-axial accelerometer (wGT3X-BT, Actigraph, Pensacola, FL, USA) were obtained from study records.

Fasted venous blood samples were directly analyzed in the Clinical Biology Laboratory of Jessa Hospital (Hasselt, Belgium) for pCRP with Cobas 8000 modular analyzer (Roche, Basel, Suisse). mCRP measurement was performed using the in-house developed aptamer based mCRP competition ELISA. Serum samples were stored at −80° C. (Biobank UBILim) [7] until analysis of mCRP.

### Statistical Analysis

SPSS version 24.0 was used. Results are described as mean±standard deviation or median (quartile 1-quartile 3), as appropriate. Proportions are expressed in percentages. pCRP and mCRP were compared between groups by using Mann-Whitney U test and a P-value<0.05 was used for significance. Spearman correlation coefficients were determined. A P-value of 50.01 was used for significance to correct for multiple testing.

### Results

### Detection of CRP in Patient Samples

As a proof of application, we applied the mCRP assay to measure the serum levels of mCRP in patients suffering from inflammation and control persons. Therefore, serum samples of healthy controls (HC), obese healthy controls (OHC) and rheumatoid arthritis (RA) patients were tested using the developed mCRP assay. RA patients (n=30) typically suffer from inflammation, and, as expected, showed a statistically significant increase (p=1.5e-05) in mCRP concentration as compared to healthy controls (n=43). The healthy obese patients (n=28) did show a low but significant increase (p=0.018) increase in mCRP as compared to the healthy controls (FIG. 6). In a second part, the mCRP assay and the DNA aptamers according to the present invention was applied to measure the serum levels in patients with COPD. Serum samples were available from 38 patients with clinically stable COPD (65±6 years, 74% male, BMI: 25.8±4.7 kg/m², FEV₁: 55.6±14.0% predicted), who had moderate-to-severe airflow obstruction, static hyperinflation and decreased diffusion capacity, moderate symptom burden, and a median of two comorbidities. Most patients were ever-smoker. Physical capacity quite well preserved, while patients were generally physically inactive (Table 1). Moreover 18 NCCP (65±6 years, 78% male, BMI: 26.4±3.0 kg/m², FEV₁: 104.8±10.2% predicted) were analyzed.

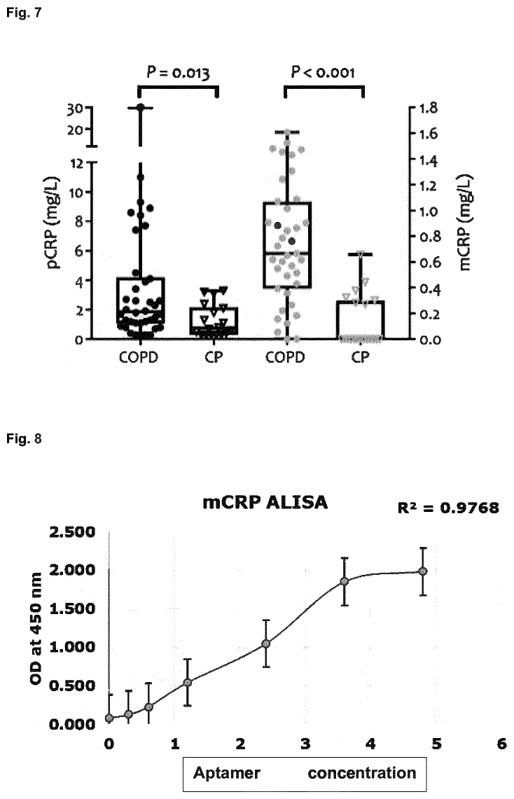

Median pCRP was higher in patients with COPD (1.85 (1.05-4.20) mg/L versus 0.75 (0.30-2.18) mg/L in NCCP; P=0.013; FIG. 7). Median mCRP levels were elevated in patients with COPD (0.66 (0.38-1.03) mg/L) versus NCCP (0.00 (0.00-0.29) mg/L; P<0.001). After excluding all participants with a pCRP>3 mg/L (COPD, n=12 and NCCP, n=3), median mCRP was still higher in patients with COPD: 0.61 (0.38-0.90) mg/L vs. (0.00 (0.00-0.28) mg/L (P<0.001).

TABLE 1
Characteristics of patients with COPD and correlation coefficients with mCRP
and pCRP

| | Patients with COPD | | Correlation | Correlation |
|---|---|---|---|---|
| Characteristics | (N = 38) | | with mCRP (ρ) | with pCRP (ρ) |
| Age (y) | 65 ± 6 | | 0.162 | 0.122 |
| Gender (N [% male]) | 28 | [74] | −0.052 | −0.215 |
| BMI (kg/m²) | 25.8 ± 4.7 | | −0.200 | 0.040 |
| Fat mass index (kg/m²)^{a} | 7.2 | (5.4-9.1) | −0.281 | −0.010 |
| Lean mass index (kg/m²)^{a} | 18.6 | (15.7-20.1) | −0.115 | 0.000 |
| FEV₁ (% predicted) | 55.6 ± 14.0 | | 0.213 | −0.017 |
| FEV₁/FVC (%) | 50.5 ± 11.9 | | 0.189 | −0.259 |
| TLC (% predicted) | 117.3 ± 16.2 | | −0.134 | −0.078 |
| RV (% predicted) | 177.7 ± 40.6 | | −0.244 | −0.293 |
| DLCO SB (% predicted) | 52.3 | (44.6-63.3) | 0.085 | −0.121 |
| GOLD Stage: I, II, III, IV (N[%]) | 3 [8], 21 [55], 13 [34], 1 [3] | | −0.210 | 0.059 |
| Hospitalization in last year: 0, 1, >1 | 32 [84], 5 [13], 1 [3] | | −0.065 | 0.158 |
| (N[%]) | | | | |
| Smoking: NS, EX, S (N[%]) | 1 [3], 21 [55], 16 [42] | | 0.255 | 0.099 |
| mMRC (points) | 1 | (0-1) | −0.322 | −0.054 |
| CAT (points) | 14 ± 6 | | −0.271 | −0.152 |
| CCI (points) | 2 | (1-3) | −0.274 | 0.086 |
| 6MWD (m) | 505 ± 76 | | 0.193 | −0.012 |
| Steps (steps/day)^{b} | 4302 | (3365-8152) | −0.003 | −0.063 |

Abbreviations and units: COPD = Chronic Obstructive Pulmonary Disease;
mCRP monomeric C-reactive Protein;
pCRP = pentameric C-reactive Protein;
BMI = Body Mass Index;
FEV₁ = Forced Expired Volume in one second;
FVC = Forced Vital Capacity;
TLC = Total Lung Capacity;
RV = Residual Volume;
DLCO SB = Diffusion capacity of the Lung for Carbon Monoxide Single Breath;
GOLD = Global initiative for Obstructive Lung Disease;
NS = Non-Smoker;
EX = EX-smoker;
S = Smoker;
mMRC = modified Medical Research Council scale for dyspnea;
CAT = COPD Assessment Test;
CCI = Charlson Comorbidity Index;
6MWD = Six-Minute Walking Distance.
y = years;
N = number;
kg = kilogram;
m = meter;
% = percentage;
^{a}sample size is n = 37 patients with COPD for this outcome;
^{b}sample size is n = 35 patients with COPD for this outcome;
ρ = correlation coefficient rho

### Example 3: Testing of Prior Art Aptamers in Serum

In order to confirm the literature observation that the aptamers published by Wang and coworkers (2011) do bind both isoforms mCRP and pCRP, we designed a competition assay for the aptamer referred in Wang comparable to the aptamer AmC2 presented in the patent application (Wang et al., 2011—Analytical and bioanalytical chemistry, vol. 401 no. 4, pg. 1309-1318).

### Materials and Methods:

pCRP was covalently bound to a COOH ELISA plate using an EDC NHS binding approach as follows. pCRP (2.5 ug/mL) was immobilized on COOH coated plates (Biomat, TN, Italy) by a covalent amine coupling using the EDC/NHS reaction. In brief, 0.4M of EDC and 0.1 M of NHS was freshly prepared and mixed in a 1:1 ratio for the activation of a COOH coated plate surface. After coating the activated surface, the remaining uncovered surfaces and unbound residual activated carboxyl groups on the plates were blocked using 5% PBS-marvel overnight followed by 1 M ethanolamine, pH 8 for 1 hour. Immobilized pCRP was in situ monomerized to mCRP by applying 10 mM NaOH for 30 min at RT. The plates were washed using 1×PBS and stored at 4° C. after 3% sucrose treatment.

A serial dilution curve was plotted for the AmC2 aptamer interaction with mCRP (1:1 binding) by applying 100 μL of freshly prepared biotin labeled aptamer in serial dilutions (4.8, 3.6, 2.4, 1.2, 0.6, 0.3, 0 mg/L) in aptamer binding buffer (ABB) (1×HEPES, 75Mm NaCl, pH 7.4). (FIG. 8)

A comparable dilution curve was plotted for the RNA aptamer (Wang) interaction with mCRP (1:1 binding) by applying 100 μL of freshly prepared biotin labeled RNA aptamer in serial dilutions (4.8, 3.6, 2.4, 1.2, 0.6, 0.3, 0 mg/L) in RNA aptamer binding buffer (RNA-ABB) (20 mM HEPES, 140 mM NaCl, 50 mM KCl, 0.1 mM DTT, and 5% glycerol at pH 6.5) as used by Wang and coworkers. (FIG. 8)

Patient sample analysis. As indicated in table 2, eight patient samples with normal or elevated pCRP serum concentration (hs pCRP ELISA) levels but negative for mCRP and 7 patient samples with a low elevated level of pCRP and positive for mCRP were analysed in duplo using either the AmC2 aptamer or the RNA aptamer (Wang) as follows. 100 μL of freshly prepared biotin labeled aptamer (AmC2 aptamer or RNA aptamer from Wang) in ABB or RNA-ABB (+RNAsin 1 U/μl, RiboLock RNase inhibitor to prevent degradation of the RNA aptamer) respectively was added to 25 μL of serum sample and incubated for 30 min at room temperature on a rotating mixer and then incubated in an mCRP coated plate for 30 min at 37° C. on a shaker at 400 rpm. After incubation, the wells were washed 3 times with ABB/RNA-ABB and incubated with Streptavidin-Horseradish peroxidase (SA-HRP) (Life technologies, Belgium) in Aptamer Conjugation Buffer (2% marvel in ABB+10% t20 or 6% BSA in RNA-ABB) for 30 min at 37° C. on a shaker at 400 rpm. After washing 3 times with ABB/ABB-RNA for 5 min, TMB substrate was added for color development, subsequently the enzyme-substrate color reaction was stopped by adding stop solution (0.18M H2SO4) to the wells. Optical density of standards and samples were measured using a Multiskan™ FC Microplate Absorbance Reader (Thermo Scientific, Belgium) at wavelength of 450 nm.

### Results and Discussion:

The dilution series obtained for both aptamers were identical confirming the equal binding of both aptamers AmC2 and RNA aptamer to mCRP immobilized onto the ELISA plate in their respective aptamer binding buffer ABB or RNA-ABB. A typical example of such a dilution series is shown in FIG. 8.

This observation confirms that the RNA aptamer binds to immobilized mCRP in buffer solutions as shown by Wang and coworkers.

In contrast to Wang et al., 2011, several literature observations, however confirmed that the aptamers of Wang also bind pCRP:
BINI et al Development of an optical RNA-based aptasensor for C-reactive protein Anal Bioanal Chem (2008) 390:1077-1086.
   Shows binding of Ca2+ stabilized pCRP to the aptamer described by Wang as a unique binder to mCRP. Additionally RNA aptamers are prone to degradation by RNases making it very difficult to use in biofluids.
Qureshi et al., Label-free RNA aptamer-based capacitive biosensor for the detection of C-reactive protein. Phys. Chem. Chem. Phys., 2010, 12, 9176-9182.
   Also these authors applied the RNA aptamer of Wang to demonstrate pCRP binding in buffer solution.
Bernard et al. Development of a bead-based aptamer/antibody detection system for C-reactive protein
   Also these authors applied the RNA aptamer of Wang to demonstrate pCRP binding in serum using a sandwich of RNA aptamer to capture pCRP in the serum and a pCRP antibody in a sandwich structure.
Centi et al. Detection of C Reactive Protein (CRP) in Serum by an Electrochemical Aptamer-Based Sandwich Assay. Electroanalysis 2009, 21, 1309-1315
   Also these authors applied the RNA aptamer of Wang to demonstrate pCRP binding in serum using a sandwich of RNA aptamer to capture pCRP in serum samples.
Linh Phung et al. Development of an Aptamer-Based Lateral Flow Assay for the Detection of C-Reactive Protein Using Microarray Technology as a Prescreening Platform ACS Comb. Sci. 2020, 22, 617-629
   These authors applied the DNA aptamer of Wang in a LFA approach to detect pCRP and as a proof used the test in pretested serum of patients clearly demonstrating the binding of pCRP in serum by the DNA aptamer of Wang.

In order to confirm these literature observations that this RNA aptamer also binds to the pentameric CRP (pCRP) isoform normally present in serum, we selected patient samples having different levels of pCRP and mCRP present in their serum. Since human serum contains ribonucleases, which may degrade the RNA aptamers, an RNAse inhibitor (RiboLock RNase inhibitor) was added as recommended by the manufacturer. The results of the obtained concentrations are shown in table 2.

TABLE 2
mCRP and pCRP values obtained for
patient serum samples.

| Sample | | | RNA apt |
|---|---|---|---|
| code | AmC2 − mCRP | pCRP | CRP |
| nummer | mg/L | mg/L | mg/L |
| JPS1 | 0 | 320 | >5 |
| JPS8 | 0 | 240 | >5 |
| JPS9 | 0 | 200 | >5 |
| JPS10 | 0 | 210 | >5 |
| JPS20 | 0 | 130 | >5 |
| JPS93 | 0 | 390 | >5 |
| JPS95 | 0 | 220 | >5 |
| UHC100 | 0 | 1.1 | 4.2 |
| JPS19 | 1.8 | 15 | >5 |
| JPS41 | 1.9 | 25 | >5 |
| JPS45 | 1.8 | 25 | >5 |
| JPS49 | 1.7 | 7.2 | >5 |
| JPS50 | 1.5 | 5.4 | >5 |
| JPS52 | 2.7 | 42 | >5 |
| JPS74 | 2.0 | 73 | >5 |

As shown in table 2, the AmC2 aptamer of the patent application is not affected by high levels of pCRP present in serum and discriminates monomeric CRP from pentameric CRP in real patient samples. Our competition assay approach calculates concentrations based on a 1:1 binding of aptamer and the mCRP target. However in case of the RNA aptamer, binding to both isoforms, the pCRP isoform may contain up to five epitopes for the RNA aptamer and as such no reliable concentration can be calculated based on this approach (conc>5 mg/L).

The RNA aptamer described by Wang and coworkers binds to both isoforms mCRP and pCRP as shown in table 2. These data indeed confirm the observations described in literature that the aptamers described by Wang also bind to the pCRP isoform. Our data clearly indicate that the aptamers described by Wang and coworkers cannot discriminate between both isoforms of CRP and hence cannot be used to determine the mCRP concentration in serum of patients as illustrated.

Taken together the competition assay demonstrates that the aptamers described by Wang and coworkers binds to both isoforms mCRP and pCRP present in serum patient samples. This confirms the findings of the literature study where the aptamer has been demonstrated to bind specifically to pCRP and that the aptamers described by Wang and coworkers can be used to reliably measure pCRP concentrations in serum of patients using different approaches.

1. Use of a DNA aptamer for the in vitro detection and/or quantification of monomeric C-reactive protein in an isolated sample of body fluid from a subject.

2. The use according to claim 1, wherein the DNA aptamer specifically binds to monomeric C-reactive protein.

3. The use according to claim 1 or 2, wherein the DNA aptamer does not bind to pentameric C-reactive protein.

4. The use according to anyone of claims 1 to 3, wherein the DNA aptamer comprises the following nucleotide sequence:
5′-kg rss ksk krs srd drk dkk rkd rwr vdv kkg dkr gtk-3′ (SEQ ID NO: 1)
wherein
k is a nucleotide selected from t or g;
r is a nucleotide selected from a or g;
s is a nucleotide selected from c or g;
d is a nucleotide selected from t or g or a;
w is a nucleotide selected from t or a; and
v is a nucleotide selected from a or c or g.

5. The use according to claim 4, wherein the DNA aptamer comprises a nucleotide sequence selected from
5-tg gcg ggt tgt gaa ggg tgg agt atg gtc gtg ttg gtt-3′ (SEQ ID NO: 2); or
5′-gg agc gcg ggg gag agt agt ggg gaa cgg tgg aga gtt-3 (SEQ ID NO: 3); or
5′-gg agg tgt gaa cgt tat gtg gta gag aga tgg gtg gtg-3′ (SEQ ID NO: 4).

6. A DNA aptamer that specifically binds to monomeric C-reactive protein and comprises the following nucleotide sequence:
5′-kg rss ksk krs srd drk dkk rkd rwr vdv kkg dkr gtk-3′ (SEQ ID NO: 1)
wherein
k is a nucleotide selected from t or g;
r is a nucleotide selected from a or g;
s is a nucleotide selected from c or g;
d is a nucleotide selected from t or g or a;
w is a nucleotide selected from t or a; and
v is a nucleotide selected from a or c or g.

7. The DNA aptamer according to claim 6 wherein the DNA aptamer does not bind to pentameric C-reactive protein.

8. The DNA aptamer according to claim 6 or 7 wherein the DNA aptamer comprises a nucleotide sequence selected from
5′-tg gcg ggt tgt gaa ggg tgg agt atg gtc gtg ttg gtt-3′ (SEQ ID NO: 2); or
5′-gg agc gcg ggg gag agt agt ggg gaa cgg tgg aga gtt-3 (SEQ ID NO: 3); or
5-gg agg tgt gaa cgt tat gtg gta gag aga tgg gtg gtg-3′ (SEQ ID NO: 4).

9. The use according to any one of claims 1 to 5 or the DNA aptamer according to any one of claims 6 to 8, wherein the DNA aptamer has a 5′ end modified by a thiol group, an amine group, a biotin, a fluorescent label, an enzyme, or a nanoparticle.

10. A method for the in vitro detection of monomeric C-reactive protein in a body fluid sample, the method comprising:
providing a DNA aptamer according to any one of claims 6 to 9;
contacting the body fluid sample with said DNA aptamer in an in vitro binding assay;
determining whether or not the DNA aptamer has bound to monomeric C-reactive protein in the body fluid sample, with binding of the DNA aptamer to monomeric C-reactive protein confirming the presence of the monomeric C-reactive protein in the body fluid sample.

11. An in vitro method for the quantification of monomeric C-reactive protein in a body fluid sample, the method comprising the method according to claim 10 wherein binding of the DNA aptamer to monomeric C-reactive protein provides an indication about the amount of mCRP in the body fluid sample.

12. The in vitro method according to claim 10 or 11, wherein the binding assay is selected from ELISA, ELASA, surface plasmon resonance assay, capillary electrophoresis-laser-induced fluorescence (CE-LIF) assay, immunohistochemistry, (nano) fluorescence-activated cell sorting (FACS), or fluorescence correlation spectroscopy.

13. A kit for the in vitro detection of monomeric C-reactive protein in a body fluid sample, said kit comprising:
a DNA aptamer according to any one of claims 6 to 9;
a blocking buffer;
an aptamer binding buffer; and
a conjugate buffer.

14. The use according to any one of claims 1 to 5 or 9, or the method according to anyone of claims 10 to 12, or the kit according to claim 13, wherein the body fluid sample is selected from blood, serum, plasma, urine, or saliva.


SEQUENCE LISTING (7 sequences)

SEQ ID NO: 1 - DNA, 38 bp, Artificial Sequence
  /note=“k is t or g” /note=“r is a or g” /note=“s is c or g” /note=“d is t or g or a” /note=“w is t or a” /note=“v is a or c or g”
kgrsskskkrssrddrkdkkrkdrwrvdvkkgdkrgtk

SEQ ID NO: 2 - DNA, 38 bp, Artificial Sequence
  AmC2 short
tggcgggttgtgaagggtggagtatggtcgtgttggtt

SEQ ID NO: 3 - DNA, 38 bp, Artificial Sequence
  AmC3 short
ggagcgcgggggagagtagtggggaacggtggagagtt

SEQ ID NO: 4 - DNA, 38 bp, Artificial Sequence
  AmC4 short
ggaggtgtgaacgttatgtggtagagagatgggtggtg

SEQ ID NO: 5 - DNA, 76 bp, Artificial Sequence
  AmC2
gcaccagcatattcgattgtggcgggttgtgaagggtggagtatggtcgtgttggttgggctagtaggtgcatcag

SEQ ID NO: 6 - DNA, 76 bp, Artificial Sequence
  AmC3
gcaccagcatattcgattgggagcgcgggggagagtagtggggaacggtggagagttgggctagtaggtgcatcag

SEQ ID NO: 7 - DNA, 76 bp, Artificial Sequence
  AmC4
gcaccagcatattcgattgggaggtgtgaacgttatgtggtagagagatgggtggtggggctagtaggtgcatcag